(19) United States
(12) Patent Application Publication
(10) Pub. No.: US 2017/0298002 A1
(43) Pub. Date: Oct. 19, 2017

(54) METHOD OF PRODUCING N-ALKYL POLYAMINES

(71) Applicants: Curza Global, LLC, Provo, UT (US); University of Utah Research Foundation, Salt Lake City, UT (US)

(72) Inventors: Ryan E. Looper, Salt Lake City, UT (US); Paul R. Sebahar, Sandy, UT (US); Travis Haussener, Midvale, UT (US)

(21) Appl. No.: 15/442,478

(22) Filed: Feb. 24, 2017

Related U.S. Application Data
(63) Continuation of application No. PCT/US2015/046810, filed on Aug. 25, 2015.
(60) Provisional application No. 62/041,588, filed on Aug. 25, 2014.

(51) Int. Cl. C07C 209/08; C07C 213/02; C07C 209/84; C07D 317/58; C07C 213/10; C07C 2601/14

(52) U.S. Cl. CPC C07C 209/08; C07D 317/58; C07C 213/10; C07C 213/02; C07C 209/84; C07C 2601/14

(57) ABSTRACT

In one embodiment, the invention presents a process for the preparation of N-alkyl polyamines that includes (i) the conversion of an amino alcohol to an aminoalkyl alkylating agent with a halo or aldehyde reactive group and (ii) the addition of amines to an amine-containing alkylating agent to make an N-alkyl polyamine.

### CROSS-REFERENCES TO RELATED APPLICATIONS

This application is a continuation of International Patent Application No. PCT/US2015/046810 filed Aug. 25, 2015, which claims the benefit of U.S. Provisional Application No. 62/041,588, filed Aug. 25, 2014, which applications are incorporated by reference in their entirety for all purposes.

### FIELD OF THE INVENTION

The present invention is directed to methods of synthesizing N-alkyl polyamine compounds in high purity. Various aspects and embodiments relate generally to intermediate compounds and to methods of preparing, purifying, and using such compounds.

### BACKGROUND OF THE INVENTION

Methods for preparing amines include, for example, U.S. Pat. Nos. 4,967,008 and 3,223,695; Int'l. Pat. Publ. No. WO 2014/016407 (i.e., U.S. Pat. Appl. Publ. No. 2015/0212132); German Pat. Publ. No. DE 3732508; Renault, J. et al. “Solid-phase combinatorial synthesis of polyamine derivatives using aminoalcohol building blocks,” Tetrahedron Lett. 2001, 42(38), 6655-58; Carboni, B. et al. “A new polyamine synthesis,” Tetrahedron Lett. 1988, 29(11), 1279-82; Cowan, J. C.; Marvel, C. S. “Ammonium salts from bromopropylamines. VI. Salts of polymeric tertiary amines,” J. Am. Chem. Soc. 1936, 58, 2277-9. See also J. Am. Chem. Soc. 1936, 52, 287; Carboni, B. et al. “Aliphatic amino azides as key building blocks for efficient polyamine syntheses,” J. Org. Chem. 1993, 58, 3736-41; and Farzaliev, V. M. et al. “Derivatives of N-alkyl(aryl)-1,2(1,3)-diazacycloalkanes. Antimicrobial properties,” Chem. Technol. Fuels Oils 2009, 45(2), 98-102.

The physiochemical properties of polyamine intermediates and products make synthesis of high-purity compounds challenging, as the products and reactants are often highly polar, difficult-to-separate compounds. The monitoring and purifications of reactions are complicated by the inability to distinguish products and side-products by NMR or LCMS. Methods to produce these polyamines are limited and typically protecting-group-intense as well as impractical for large-scale synthesis. See, e.g. Bergeron, R. J. et al. “Reagents for the Stepwise Functionalization of Spermidine, Homospermidine and Bis(3-aminopropyl)amine.” J. Org. Chem. 1984, 49, 2997-3001; Saab, N. H. et al. “Synthesis and evaluation of unsymmetrically substituted polyamine analogues as modulators of human spermidine/spermine-N1-acetyltransferase (SSAT) and as potential antitumor agents.” J. Med. Chem. 1993, 36, 2998-3004; Bergeron, R. J. et al. “Synthetic Polyamine Analogues as Antineoplastics.” J. Med. Chem. 1988, 31, 1183-90; Renault, S. C. et al. “Solid-phase Organic Synthesis of Unnatural Polyamine Analogues Bearing a Dansyl or Acridine Moiety.” Pharm. Pharmacol. Commun. 1999, 5, 151-57.

For a scalable process, a protecting-group-free synthesis of polyamines would be advantageous. A protecting group-free synthesis with few synthetic steps would likely be more efficient for making various polyamine analogs because of the lack of protection and deprotection steps. Avoiding chromatographic purification would also be helpful for successful scale-up because of its high cost at large scale. The inventive process provides an improved method for addressing at least these problems. In preferred aspects, the inventive process solves one or more of the problems of simplifying the separation or purification of the product, avoiding protection/deprotection steps, and improving yield.

### BRIEF SUMMARY OF THE INVENTION

In one embodiment, the invention presents a process for the preparation of N-alkyl polyamines that includes (i) the conversion of an amino alcohol to an aminoalkyl alkylating agent with a halo or aldehyde reactive group and (ii) the addition of amines to an amine-containing alkylating agent to make an N-alkyl polyamine.

### BRIEF DESCRIPTION OF THE DRAWINGS

FIG. 1. (A) An aspect of the claimed method, in which a N-isobutyl norspermidine is prepared. (B) The use of the N-isobutyl norspermidine to prepare a di-(N-alkyl polyamino) compound.

FIG. 2. A general method for preparation and use of a alkylamino alkylating agent comprising a halo group.

FIG. 3. Exemplary substrates for preparation according to the disclosed methods.

The accompanying drawings are discussed in reference to the numerals provided therein so as to enable one skilled in the art to practice embodiments of the present invention. The skilled artisan will understand, however, that the inventions described below can be practiced without employing these specific details, or that they can be used for purposes other than those described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

### DETAILED DESCRIPTION OF THE INVENTION

It will be appreciated that reference throughout this specification to aspects, features, advantages, or similar language does not imply that all of the aspects and advantages that may be realized with the present invention should be or are in any single embodiment of the invention. Rather, language referring to the aspects and advantages is understood to mean that a specific aspect, feature, advantage, or characteristic described in connection with an embodiment is included in at least one embodiment of the present invention. Thus, discussion of the aspects and advantages, and similar language, throughout this specification may, but does not necessarily, refer to the same embodiment.

The described aspects, features, advantages, and characteristics of the invention may be combined in any suitable manner in one or more further embodiments. Furthermore, one skilled in the relevant art will recognize that the invention may be practiced without one or more of the specific aspects or advantages of a particular embodiment. In other instances, additional aspects, features, and advantages may be recognized and claimed in certain embodiments, but may not be present in all embodiments of the invention.

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety, including U.S. Pat. Appl. No. 62/001,604 (docket no. 96175-909657-000451US). In case of conflict, the present specification, including these definitions, will control.

The terms “a,” “an,” or “the” as used herein not only includes aspects with one member, but also includes aspects with more than one member. For example, an embodiment including “a polyamine compound and an excipient” should be understood to present certain aspects with at least a second polyamine compound, at least a second excipient, or both.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” When the quantity “X” only includes whole-integer values (e.g., “X carbons”), “about X” indicates from (X−1) to (X+1). In this case, “about X” as used herein specifically indicates at least the values X, X−1, and X+1.

When the term “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.” However, when the modifier “about” is applied to describe only the end of a range or only a later value in a set of values, it applies only to that value or that end of the range. Thus, the range “about 2 to 10” is the same as “about 2 to about 10,” but the range “2 to about 10” is not.

The term “acyl” as used herein includes an alkanoyl, aroyl, heterocycloyl, or heteroaroyl group as defined herein. Examples of acyl groups include, but are not limited to, acetyl, benzoyl, and nicotinoyl.

The term “alkanoyl” as used herein includes an alkyl-C(O)— group wherein the alkyl group is as defined herein. Examples of alkanoyl groups include, but are not limited to, acetyl and propanoyl.

The term “agent” as used herein includes a compound or mixture of compounds that, when added to a composition, tend to produce a particular effect on the composition's properties. For example, a composition comprising a thickening agent is likely to be more viscous than an otherwise identical comparative composition that lacks the thickening agent.

The term “alkenyl” as used herein includes a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. When the indicated number of carbon atoms is 1, then the C₁ alkenyl is double bonded to a carbon (i.e., a carbon analog to an oxo group). In certain aspects, the chain includes 1 to 12, about 2 to 15, about 2 to 12, about 2 to 8, or about 2 to 6 carbon atoms. Examples of an alkenyl group may include, but are not limited to, ethenyl (i.e., vinyl), allyl, propenyl, butenyl, crotyl, pentenyl, hexenyl, heptenyl, octenyl, nonenyl, decenyl, dodecenyl, cyclopentenyl, cyclohexenyl, 2-isopentenyl, allenyl, butadienyl, pentadienyl, 3-(1,4-pentadienyl), and hexadienyl.

In some aspects, an alkenyl group is unsubstituted. In some aspects, an alkenyl group is optionally substituted. When optionally substituted, one or more hydrogen atoms of the alkenyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom substituent on the carbon-carbon double bond is replaced by a hydroxy, amino, or thio group.

The term “alkyl” as used herein includes an aliphatic hydrocarbon chain that may be straight chain or branched. The chain may contain an indicated number of carbon atoms: For example, C₁-C₁₂ indicates that the group may have from 1 to 12 (inclusive) carbon atoms in it. If not otherwise indicated, an alkyl group about 1 to about 20 carbon atoms. In some aspects, alkyl groups have 1 to about 12, 1 to about 10, 1 to about 8, 1 to about 6, or 1 to about 4 carbon atoms in the chain. In another aspect, alkyl groups (“lower alkyl”) have 1 to about 6, 1 to 5, 1 to 4, or 1 to 3 carbon atoms in the chain. Examples may include, but are not limited to, methyl, ethyl, propyl, isopropyl (iPr), 1-butyl, 2-butyl, isobutyl (iBu), tert-butyl, pentyl, 2-methylbutyl, 1,1-dimethylpropyl, hexyl, heptyl, octyl, nonyl, decyl, docecyl, cyclopentyl, or cyclohexyl. In some aspects, an alkyl group can exclude methyl (e.g., 2 to 6 carbon atoms in the chain).

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, the alkyl group is unsubstituted or not optionally substituted.

The term “alkoxy” as used herein includes a straight or branched chain saturated or unsaturated hydrocarbon containing at least one oxygen atom in an ether group (e.g., EtO—). The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy include, but are not limited to, methoxy, ethoxy, isopropoxy, butoxy, n-pentoxy, isopentoxy, neopentoxy, and hexoxy.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom alpha to the ether oxygen is replaced by a hydroxy, amino, or thio group. In some aspects, the alkoxy group is unsubstituted or not optionally substituted.

The term “alkynyl” as used herein includes a straight, branched, or cyclic hydrocarbon containing at least one carbon-carbon triple bond. Examples may include, but are not limited to, ethynyl, propargyl, propynyl, butynyl, pentynyl, hexynyl, heptynyl, octynyl, nonynyl, decynyl, or decynyl.

An alkynyl group can be unsubstituted or optionally substituted. When optionally substituted, one or more hydrogen atoms of the alkynyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no sp hydrogen atom substituent is replaced by a hydroxy, amino, or thio group. In some aspects, the alkynyl group is unsubstituted or not optionally substituted.

The term “aroyl” as used herein includes an aryl-CO— group wherein aryl is as defined herein. Examples include, but are not limited to, benzoyl, naphth-1-oyl and naphth-2-oyl.

The term “aryl” as used herein includes cyclic aromatic carbon ring systems containing from 6 to 18 carbons. Examples of an aryl group include, but are not limited to, phenyl, naphthyl, anthracenyl, tetracenyl, biphenyl and phenanthrenyl.

An aryl group can be unsubstituted or optionally substituted. When optionally substituted, one or more hydrogen atoms of the aryl group (e.g., from 1 to 5, from 1 to 2, or 1) may be replaced with a moiety independently selected from the group consisting of alkyl, cyano, acyl, halo, hydroxy, alkoxy, amino, alkylamino, acylamino, thio, and alkylthio. In some aspects, the aryl group is unsubstituted or not optionally substituted.

The term “arylalkyl” or “aralkyl” as used herein includes an alkyl group as defined herein where at least one hydrogen substituent has been replaced with an aryl group as defined herein. Examples include, but are not limited to, benzyl, 1-phenylethyl, 4-methylbenzyl, and 1,1,-dimethyl-1-phenylmethyl.

A group can be unsubstituted or optionally substituted as per its component parts. For example, but without limitation, the aryl group of an arylalkyl group can be substituted, such as in the arylalkyl group 4-methylbenzyl. In some aspects, and preferably, the group is unsubstituted or not optionally substituted, especially if it includes a defined substituent, such as a hydroxyalkyl or alkylaminoalkoxy group.

The linking term “comprising” or “comprise” as used herein is not closed. For example, “a composition comprising A” must include the component A, but it may incorporate one or more other components (e.g., B; B and C; and the like).

The term “cycloalkyl” as used herein includes a cyclic hydrocarbon group that may contain an indicated number of carbon atoms: For example, C₃-C₁₂ indicates that the group may have from 3 to 12 (inclusive) carbon atoms in it. If not otherwise indicated, a cycloalkyl group includes about 3 to about 20 carbon atoms. In some aspects, cycloalkyl groups have 3 to about 12 carbon atoms in the group. In another aspect, cycloalkyl groups have 3 to about 7 carbon atoms in the group. Examples may include, but are not limited to, cyclopropyl, cyclobutyl, cyclopentyl, cyclohexyl, 4,4-dimethylcyclohexyl, and cycloheptyl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, a substituted cycloalkyl group can incorporate an exo- or endocyclic alkene (e.g., cyclohex-2-en-1-yl). In some aspects, a cycloalkyl group is unsubstituted or not optionally substituted.

The term “effective amount” or “effective dose” as used herein includes an amount sufficient to achieve the desired result and accordingly will depend on the ingredient and its desired result. Nonetheless, once the desired effect is identified, determining the effective amount is within the skill of a person skilled in the art.

As used herein, “fluoroalkyl” includes an alkyl group wherein the alkyl group includes one or more fluoro-substituents. Examples include, but are not limited to, trifluoromethyl.

As used herein, “geminal” substitution includes two or more substituents that are directly attached to the same atom. An example is 3,3-dimethyl substitution on a cyclohexyl or spirocyclohexyl ring.

As used herein, “halo” or “halogen” includes fluoro, chloro, bromo, or iodo. Preferably, for a N-(haloalkyl) alkylamine, “halo” includes bromo or chloro.

An alkylene “halide” as described herein is a haloalkyl group. For example, N-alkyl propylene halide is equivalent to N-alkyl halopropane (i.e., comprising a C—X bond, where X is halogen). In contrast, a salt with a halide counterion is, e.g., an alkylammonium bromide (i.e., a A⁺ cation and an X⁻ anion).

The term “heteroaryl” includes mono and bicyclic aromatic groups of about 4 to about 14 ring atoms (e.g., 4 to 10 or 5 to 10 atoms) containing at least one heteroatom. Heteroatom as used in the term heteroaryl refers to oxygen, sulfur and nitrogen. A nitrogen atom of a heteroaryl is optionally oxidized to the corresponding N-oxide. Examples include, but are not limited to, pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, and 1,2,4-triazinyl, benzothiazolyl.

A heteroaryl group can be unsubstituted or optionally substituted. When optionally substituted, one or more hydrogen atoms of the heteroaryl group (e.g., from 1 to 5, from 1 to 2, or 1) may be replaced with a moiety independently selected from the group consisting of alkyl, cyano, acyl, halo, hydroxy, alkoxy, amino, alkylamino, acylamino, thio, and alkylthio. In some aspects, the heteroaryl group is unsubstituted or not optionally substituted.

The term “heteroaroyl” as used herein includes a heteroaryl-C(O)— group wherein heteroaryl is as defined herein. Heteroaroyl groups include, but are not limited to, thiophenoyl, nicotinoyl, pyrrol-2-ylcarbonyl, and pyridinoyl.

The term “heterocycloyl” as used herein includes a heterocyclyl-C(O)— group wherein heterocyclyl is as defined herein. Examples include, but are not limited to, N-methyl prolinoyl and tetrahydrofuranoyl.

As used herein, “heterocyclyl” includes a non-aromatic saturated monocyclic or multicyclic ring system of about 4 to about 10 ring atoms (e.g., 5 to about 8 ring atoms, or 5 to about 6 ring atoms), in which one or more of the atoms in the ring system is an element or elements other than carbon, e.g., nitrogen, oxygen or sulfur. A heterocyclyl group optionally comprises at least one sp²-hybridized atom (e.g., a ring incorporating an carbonyl, endocyclic olefin, or exocyclic olefin). In some embodiments, a nitrogen or sulfur atom of the heterocyclyl is optionally oxidized to the corresponding N-oxide, S-oxide or S,S-dioxide. Examples of monocycylic heterocyclyl rings include, but are not limited to, piperidinyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, and tetrahydrothiopyranyl.

A heterocycyl group can be unsubstituted or optionally substituted. When optionally substituted, one or more hydrogen atoms of the group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, a substituted heterocycyl group can incorporate an exo- or endocyclic alkene. In some aspects, the heterocycyl group is unsubstituted or not optionally substituted.

As used herein, the term “hydroxyalkyl” includes an alkyl group where at least one hydrogen substituent has been replaced with an alcohol (—OH) group. In certain aspects, the hydroxyalkyl group has one alcohol group. In certain aspects, the hydroxyalkyl group has one or two alcohol groups, each on a different carbon atom. In certain aspects, the hydroxyalkyl group has 1, 2, 3, 4, 5, or 6 alcohol groups. Examples may include, but are not limited to, hydroxymethyl, 2-hydroxyethyl, and 1-hydroxyethyl.

When any two substituent groups or any two instances of the same substituent group are “independently selected” from a list of alternatives, the groups may be the same or different. For example, if R^{a} and R^{b} are independently selected from the group consisting of alkyl, fluoro, amino, and hydroxyalkyl, then a molecule with two R^{a} groups and two R^{b} groups could have all groups be alkyl group (e.g., four different alkyl groups). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different).

As used herein, “polyamine” includes a compound that has at least two amine groups, which may be the same or different. The amine group may be a primary amine, a secondary amine, a tertiary amine, or quaternary ammonium salt. Examples may include, but are not limited to, 1,3-diaminopropane, 1,4-diaminobutane, hexamethylenediamine, dodecan-1,12-diamine, spermine, spermidine, norspermine, and norspermidine.

As used herein, “or” should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, “spirocycloalkyl” as used herein includes a cycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a cyclopropyl ring incorporating the carbon to which R¹ and R² were bonded, this would be a spirocycloalkyl group (i.e., spirocyclopropyl).

As used herein, “spiroheterocyclyl” as used herein includes a heterocycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a pyrrolidine ring incorporating the carbon to which R¹ and R² were bonded, this would be a spiroheterocyclyl group.

### Method

In one embodiment, the invention sets forth a method of preparing an N-alkyl polyamine, wherein the method comprises the steps:

reacting an aminoalkyl alkylating agent in a reaction mixture comprising an excess amount of a polyaminoalkane to produce a N-alkyl polyamine, wherein the aminoalkyl alkylating agent comprises (i) a secondary or tertiary amino group and (ii) a halo or aldehyde group; and wherein the N-alkyl polyamine has from 5 to 30 carbon atoms; and
distilling a crude product comprising the N-alkyl polyamine to provide a purified N-alkyl polyamine.

In one aspect, amino alcohols present several advantages as a starting material for the inventive process, including: 1) options for synthetic manipulation of the amine without affecting the alcohol functionality on the chain (e.g., selective monoalkylation of the amine by controlled reductive amination); and 2) a leaving group synthon (i.e., the hydroxyl) that can be activated for displacement later. Direct alkylation of a diamine typically produced bis-alkylated impurities that decreased the efficiency of the reaction and purification. A further advantage is the low cost and ready availability in large quantities (>20 kg) of some amine alcohols (e.g., 3-amino-1-propanol).

In one aspect, the N-alkyl polyamine has from 20 to 30 carbon atoms. In a more specific aspect, the N-alkyl polyamine has from 20 to 26 carbon atoms.

In an alternative aspect, the N-alkyl polyamine has from 5 to 20 carbon atoms. In a more specific aspect, the N-alkyl polyamine has from 10 to 20 carbon atoms. In an alternative more specific aspect, the N-alkyl polyamine has from 5 to 15 carbon atoms. In an alternative more specific aspect, the N-alkyl polyamine has from 10 to 15 carbon atoms.

In one aspect, the step of reacting the aminoalkyl alkylating agent is performed at a temperature from −78° C. to 150° C. (e.g., about −78° C., about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., or about 35° C.). In a more specific aspect, the step of reacting the aminoalkyl alkylating agent is performed at a temperature from −78° C. to 120° C. In a more specific aspect, the step of reacting the aminoalkyl alkylating agent is performed at a temperature from −78° C. to 100° C. In a more specific aspect, the step of reacting the aminoalkyl alkylating agent is performed at a temperature from −25° C. to 100° C. In a more specific aspect, the step of reacting the aminoalkyl alkylating agent is performed at a temperature from −10° C. to 100° C. In a more specific aspect, the step of reacting the aminoalkyl alkylating agent is performed at a temperature from 0° C. to 100° C. In a more specific aspect, the step of reacting the aminoalkyl alkylating agent is performed at a temperature from 0° C. to 80° C. In a more specific aspect, the step of reacting the aminoalkyl alkylating agent is performed at a temperature from 0° C. to 60° C. In a more specific aspect, the step of reacting the aminoalkyl alkylating agent is performed at a temperature from 0° C. to 40° C. (e.g., at room temperature, ca. 20° C.). In a more specific aspect, the step of reacting the aminoalkyl alkylating agent is performed at a temperature from 10° C. to 25° C. In a more specific aspect, the step of reacting the aminoalkyl alkylating agent is performed at a temperature from about 0° C. to 20° C.

In one aspect, the step of distilling the crude product is performed at below atmospheric pressure. In a more specific aspect, the step of distilling the crude product is performed at a pressure from 10 mm Hg to 25 mm Hg. In an alternative more specific aspect, the step of distilling the crude product is performed at a pressure from 1 mm Hg to 10 mm Hg. In an alternative more specific aspect, the step of distilling the crude product is performed at a pressure from 0.01 mm Hg to 1 mm Hg.

In one preferred aspect, the present invention ensures that the excess amine reacted with the aminoalkyl alkylating agent has a boiling point that is low enough to allow easy separation of it from the desired N-alkyl polyamine product under the distillation conditions. In one aspect, the distilled product has a boiling point at least 20° C. higher than the excess amine (e.g., diaminoalkane). In one aspect, the desired product has a boiling point at least 25° C., at least 30° C., at least 35° C., at least 40° C., at least 45° C., at least 50° C., at least 60° C., or at least 75° C. higher than the excess amine (e.g., the excess diaminoalkane, such as norspermine or norspermidine).

In one preferred aspect, the present invention ensures that any significant byproducts and impurities of the reaction (e.g., overalkylation products of high molecular weight compared to the desired product) have a boiling point that is high enough to allow easy separation of them from the desired N-alkyl polyamine product under the distillation conditions. In one aspect, the significant byproducts and impurities are not volatile under the distillation conditions. In one aspect, the desired product has a boiling point at least 20° C. lower than such high-boiling byproducts and impurities. In one aspect, the desired product has a boiling point at least 25° C., at least 30° C., at least 35° C., at least 40° C., at least 45° C., at least 50° C., at least 60° C., or at least 75° C. than such high-boiling byproducts and impurities.

In one aspect, the step of reacting the aminoalkyl alkylating agent includes no added solvent. In an alternative aspect, the step of reacting the aminoalkyl alkylating agent includes added solvent.

In one aspect, the aminoalkyl alkylating agent is of the formula

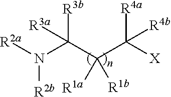

wherein each R substituent is an independently selected hydrogen, alkyl, alkoxy alkenyl, or alkynyl group, with the proviso that the R² substituents are not hydrogen; and wherein X is —CHO.

In one aspect, the aminoalkyl alkylating agent is of the formula

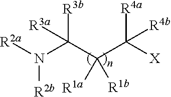

wherein each R substituent is an independently selected hydrogen, alkyl, alkoxy alkenyl, or alkynyl group; wherein at least one R² substituent is not hydrogen; and wherein X is a halo group.

In a more specific aspect, at least one R^{1a} and R^{1b} are alkyl. In a more specific aspect, at least one R^{1a} and R^{1b} are methyl. In an alternative more specific aspect, R^{1a} and R^{1b} are hydrogen. In an alternative more specific aspect, R^{1a} and R^{1b} are joined to form a spirocyclopropyl ring.

In a more specific aspect, R^{2a} is an alkyl and R^{2b} is hydrogen. In an alternative more specific aspect, R^{2a} is an alkyl and R^{2b} is an alkyl.

In a more specific aspect, R^{3a} and R^{3b} are hydrogen.

In a more specific aspect, R^{4a} and R^{4b} are hydrogen.

In a more specific aspect, X is chloro, bromo, or iodo. In an alternative more specific aspect, X is a chloro.

In one aspect, the aminoalkyl alkylating agent is an N-alkyl propylene halide or aldehyde. In an alternative aspect, the aminoalkyl alkylating agent is an N-alkyl butylene halide or aldehyde. In an alternative aspect, the aminoalkyl alkylating agent is an N-alkyl ethylene halide or aldehyde. In an alternative aspect, the aminoalkyl alkylating agent is an N-alkyl pentylene halide or aldehyde. In an alternative aspect, the aminoalkyl alkylating agent is an N-alkyl hexylene halide or aldehyde.

In one aspect, the N-alkyl group is butyl. In an alternative aspect, the N-alkyl group is isobutyl. In an alternative aspect, the N-alkyl group is hexyl. In an alternative aspect, the N-alkyl group is (cyclohexyl)methyl. In an alternative aspect, the N-alkyl group is octyl. In an alternative aspect, the N-alkyl group is isopropyl. In an alternative aspect, the N-alkyl group is methyl. In an alternative aspect, the N-alkyl group is ethyl. In an alternative aspect, N-alkyl group is cyclohexyl. In an alternative aspect, the N-alkyl group is prenyl. In an alternative aspect, the N-alkyl group is propargyl. In an alternative aspect, the N-alkyl group is cyclopropyl.

In one aspect, the halide or halo is chloride. In an alternative aspect, the halide or halo is bromide.

In one aspect, the aminoalkyl alkylating agent is a crystalline salt with a halide counterion.

In one aspect, the polyaminoalkane is spermidine. In an alternative aspect, the polyaminoalkane is norspermidine.

In one aspect, the excess amount of diamine is about 2 or at least 2 equivalents (e.g., about 2, 2.5, 3, 3.5, 4, 4.5, or 5 equivalents). In an alternative aspect, the excess amount is about 5 or at least 5 equivalents (e.g., about 5, 6, 7, or 8 equivalents). In an alternative aspect, the excess amount is about 8 or at least 8 equivalents (e.g., about 8, 9, 10, 11, or 12 equivalents). In an alternative aspect, the excess amount is about 12 or at least 12 equivalents (e.g., about 12, 13, 14, 15, or 16 equivalents). In an alternative aspect, the excess amount is about 16 or at least 16 equivalents (e.g., about 16, 17, 18, 19, or 20 equivalents). In an alternative aspect, the excess amount is about 10 to 20 equivalents (e.g., about 10, 11, 12, 13, 14, 15, 16, 17, 18, 19, or 20 equivalents). In an alternative aspect, the excess amount is about 20 or at least 20 equivalents (e.g., about 20, 21, 22, 23, or 24 equivalents). In an alternative aspect, the excess amount is about 24 or at least 24 equivalents (e.g., about 24, 25, 26, 27, or 28 equivalents). In an alternative aspect, the excess amount is about 28 or at least 28 equivalents (e.g., about 28, 29, 30, 31, or 32 equivalents). In an alternative aspect, the excess amount is about 32 or at least 32 equivalents (e.g., about 32, 33, 34, 35, or 36 equivalents). In an alternative aspect, the excess amount is about 36 or at least 36 equivalents (e.g., about 36, 37, 38, 39, or 40 equivalents). In an alternative aspect, the excess amount is about 40 or at least 40 equivalents (e.g., about 40, 41, 42, 43, 44, 45, 46, 47, 48, 49, or 50 equivalents). In an alternative aspect, the excess amount is about 50 or at least 50 equivalents (e.g., about 50, 51, 52, 53, 54, 55, 60, 65, 70, or 75 equivalents).

In one aspect, the method further comprises a step of distilling the crude product to produce a purified diaminoalkane. In one aspect, the distilling step is under reduced pressure. In one aspect, the excess diaminoalkane is at least partially removed by aqueous extraction.

In one aspect, the method further comprises reusing the purified diaminoalkane as a substrate for alkylation.

In one aspect, the method further comprises a step of reacting an aminoalkyl alcohol precursor to produce the aminoalkyl alkylating agent, e.g., as a crystalline salt. In one aspect, the step is the conversion of an alcohol to a halide (e.g., to a bromide). In one aspect, the step comprises treatment with an acidic solution of a nucleophile (e.g., an hydrobromic acid solution, such as concentrated aqueous HBr at reflux). In one aspect, the crude salt product is prepared by distillation of the volatile reagents. In one aspect, the crude crystalline product is purified by recrystallization (e.g., with MeOH/Et₂O or isopropanol).

In one aspect, the method further comprises a step of reacting a primary aminoalkyl alcohol with an alkyl aldehyde or a cycloalkylmethyl aldehyde to produce the aminoalkyl alcohol precursor (e.g., by condensation to produce an imine and reduction of the imine to an amine, e.g., with sodium borohydride in water). In an alternative more specific aspect, the method further comprises a step of reacting a secondary aminoalkyl alcohol with an alkyl aldehyde or a cycloalkylmethyl aldehyde to produce the aminoalkyl alcohol precursor. In some aspects, the step is a selective reduction that produces a secondary amine.

In one aspect, the method further comprises a step of reacting the purified N-alkyl polyamine with an aldehyde or halide (preferably, an aryl, heteroaryl, or phenyl group with a haloalkyl or aldehyde substituent) to produce an oligomeric polyamine. In a more specific aspect, the step is a reductive amination (e.g., with sodium borohydride in methanol).

In one further aspect, the method further comprises a step of reacting the purified N-alkyl polyamine with a polyaldehyde or polyhalide (preferably, a phenyl group with haloalkyl or aldehyde substituents) to produce an oligomeric polyamine. In a more specific aspect, the oligomeric polyamine is a compound set forth in U.S. Appl. Nos. 62/001,604 (docket no. 96175-909657-000451US) or Ser. No. 14/076,143 (i.e., U.S. Pat. No. 8,853,278). In an alternative more specific aspect, the oligomeric polyamine is a compound set forth in U.S. application Ser. No. 14/507,701 (i.e., U.S. Pat. Appl. Publ. No. 2015/0038512).

In a more specific aspect, the oligomeric polyamine is a polyamine compound selected from the group including

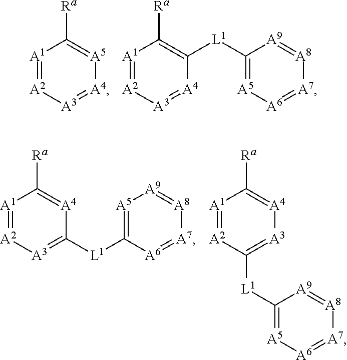

and a salt thereof; wherein:

each R^{a} is a member independently selected from the including

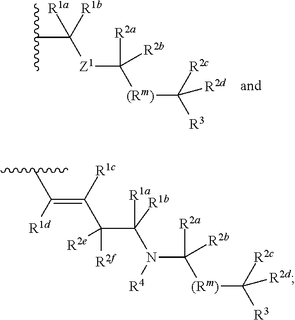

A¹, A², A³, A⁴, A⁵, A⁶, A⁷, A⁸, and A⁹ are each an Aⁿ member independently selected from the group including N, CR^{a}, and CR⁵; or, alternatively, a pair of adjacent Aⁿ members join to form an independently selected aryl, cycloalkyl, heterocyclyl, or heterocycloaryl ring that is fused with an Aⁿ ring at the pair's Aⁿ ring positions; wherein at least one Aⁿ member and at most five Aⁿ members are an independently selected CR^{a};

each R^{1a}, R^{1b}, R^{1c}, and R^{1d} is a member independently selected from the group including hydrogen, fluoro, alkyl, and fluoroalkyl; or, alternatively, an R^{1a} and an R^{1b} join to form an oxo group;

each R^{2a}, R^{2b}, R^{2c}, R^{2d}, R^{2e}, and R^{2f} is a member independently selected from the group including hydrogen, alkyl, fluoroalkyl, alkenyl, alkynyl, aryl, heteroaryl, arylalkyl, and heteroarylalkyl; alternatively, a pair of R² members from the same R^{a} group independently selected from R^{2a} and R^{2b}, R^{2c} and R^{2d}, or R^{2e} and R^{2f} join to form a member independently selected from the group including spirocycloalkyl, spiroheterocycyl, and oxo; or, alternatively, an R^{2a} and an R^{2c} from the same R^{a} group join to form a ring independently selected from the group including cycloalkyl and heterocycyl;

each R^{m} is a member independently selected from the group including —CR^{2a}R^{2b}—, —CR^{2c}R^{2d}—, —C(R^{2a})═(R^{2b})—, —CC—, and —C(R^{2a})(R^{2b})-L²-C(R^{2c})(R^{2d})—;

each m is an integer independently selected from 1 to 20;

each L¹ and L² is a member independently selected from the group including a bond, —O—, —C(O)O—, —NR⁴—, —NR⁴C(O)—, and —C(O)NR⁴—;

each R³ is a member independently selected from the group including —Z¹—R⁴, —Z¹—Y¹—R⁴, —Z¹—Y¹—Y²—R⁴, and —Z¹—Y¹—Y²—Y³—R⁴;

each R⁴ is a member independently selected from the group including hydrogen, alkyl, fluoroalkyl, alkenyl, alkynyl, aryl, cycloalkyl, heteroaryl, arylalkyl, cycloalkylalkyl, and heteroarylalkyl; or, alternatively, for an —N(R⁴)₂ group, one of the two R⁴ in the group is a member selected from the group consisting of —(CO)OR^{6a}, —(CO)N(R^{6a})(R^{6b}), and —C(NR^{6a})N(R^{6b})(R^{6c}); or, alternatively, for an —N(R⁴)₂ group, the two R⁴ groups join to form a heterocyclic ring;

each R⁵ is a member independently selected from the group including hydrogen, alkyl, hydroxyl, alkoxy, aminoalkoxy, alkylamino, alkylaminoalkoxy, alkenyl, alkynyl, aryl, aryloxy, arylamino, cycloalkyl, cycloalkoxy, cycloalkylalkoxy, cycloalkylamino, cycloalkylalkylamino, heterocyclyl, heterocycyloxy, heterocycylamino, halo, haloalkyl, fluoroalkyloxy, heteroaryl, heteroaryloxy, heteroarylamino, arylalkyl, arylalkyloxy, arylalkylamino, heteroarylalkyl, heteroarylalkyloxy, heteroarylalkylamino, hydroxyalkyl, aminoalkyl, and alkylaminoalkyl;

each Y¹, Y², and Y³ is an independently selected group of Formula IA:

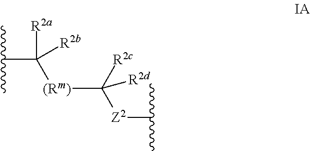

each Z¹ and Z² is a member independently selected from the group including N(R⁴)— and —O—; and

each R^{6a}, R^{6b}, and R^{6C} is a member independently selected from the group including hydrogen, alkyl, fluoroalkyl, alkenyl, alkynyl, aryl, heteroaryl, cycloalkyl, arylalkyl, heteroarylalkyl, and cycloalkylalkyl; or, alternatively, two R⁶ members R^{6a} and R^{6b} or R^{6a} and R^{6c} join to form a heterocycyl ring; and wherein the polyamine compound comprises at least two primary or secondary amino groups.

In a more specific aspect, the oligomeric polyamine is

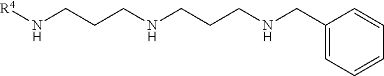

or a salt thereof, and
wherein R⁴ is hydrogen or alkyl

In a more specific aspect, the oligomeric polyamine is

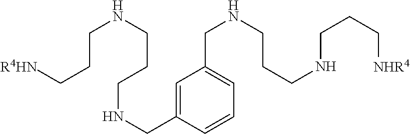

or a salt thereof, and
wherein R⁴ is hydrogen or alkyl

In a more specific aspect, the oligomeric polyamine is

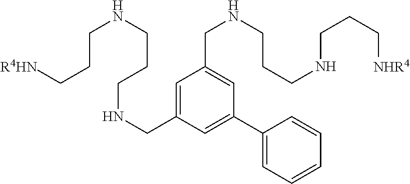

or a salt thereof, and
wherein R⁴ is hydrogen or alkyl.

In one aspect, the invention sets forth a composition for use in a method that is set forth herein.

### EXAMPLES

### General Experimental Conditions

Unless otherwise noted, materials were obtained from commercial sources and used without purification; otherwise, materials were purified according to Purification of Laboratory Chemicals. All reactions requiring anhydrous conditions were performed under a positive pressure of nitrogen using flame-dried glassware. Methanol (MeOH) was distilled over magnesium prior to its usage. Diaminopropane is highly toxic and should be handled with great care. Any volatile polyamine synthesized should also be regarded as toxic and should be handled with care and always stored under N₂ due to reactivity with O₂ and CO₂. Distillations were carried out under reduced pressure with a sodium bicarbonate (NaHCO₃) scrubber for distillations involving HBr and a citric acid scrubber for any distillations involving amines. Yields were calculated for material judged homogeneous by thin-layer chromatography and ¹H NMR. Thin-layer chromatography was performed on silica plates eluting with the solvents indicated and visualized by a 254 nm UV lamp or permanganate stain.

¹H NMR spectra were recorded at 500 or 300 MHz as indicated. The chemical shifts (δ) of proton resonances are reported relative to the deuterated solvent peak: 7.26 for CDCl₃ and 4.79 for H₂O using the following format: chemical shift [multiplicity (s=singlet, d=doublet, dd=doublet of doublets, t=triplet, q=quartet, pent=pentet, hex=hextet, sept=septet, oct=octet, non=nonet m=multiplet), coupling constant(s) (J in Hz), integral]. ¹³C NMR spectra were recorded at 125 MHz. The chemical shifts of carbon resonances are reported relative to the deuterated solvent peak: 77.00 (first line) for CDCl₃. Certain carbon experiments conducted with the VXR500 MHz NMR contained an artifact peak between 170.0-174.0 ppm. Mass spectra were obtained by ESI+/APCI for LRMS or ESI+/APCI-TOF for HRMS.

### Polyamine Naming

These examples include a simplified naming system for the polyamine side chains that were synthesized. Hence, derivatives may be named as “[side chain group] [polyamine group].” For example, the compound N¹-(3-aminopropyl)-N³-butylpropane-1,3-diamine would alternatively be referred to as “n-butyl norspermidine.” The compound N¹-(3-(isobutylamino)-propyl)butane-1,4-diamine would alternatively be referred to as “i-butyl spermidine” (or, equivalently, “iso-butyl spermidine” or “isobutyl spermidine”).

### Example 1: Synthesis of Isobutyl Norspermidine

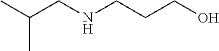

### 3-(Isobutylamino)propan-1-ol

3-Amino-1-propanol (35.4 g, 0.58 mol, 1.0 equiv.) and 3 Å mol. sieves were placed in a round bottomed flask. The solution was cooled to 0° C. (ice/water), and isobutyraldehyde (41.8 g, 0.58 mol, 1.0 equiv.) was added over the span of 20 min. The reaction was left to warm and stirred for 8 h. Sodium borohydride (11.0 g, 0.29 mol, 0.5 equiv.) in water (100 mL) was added slowly to the reaction mixture. After bubbling had ceased, the solution was extracted with EtOAc (2×200 mL), dried over Na₂SO₄, filtered, and concentrated under reduced pressure to afford the 3-(isobutylamino)propan-1-ol as a yellow oil (65.8 g, 97%). ¹H NMR (300 MHz, CDCl₃) δ ppm 3.79 (t, J=5.1 Hz, 2H), 2.84 (t, J=5.7 Hz, 2H), 2.40 (d, J=6.6 Hz, 2H), 1.70-1.62 (m, 3H), 0.88 (d, J=6.9 Hz, 6H). ¹³C NMR (125 MHz, CDCl₃) δ ppm 65.0, 58.2, 50.7, 30.8, 28.6, 21.0.

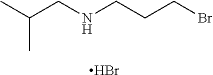

### 3-Bromo-N-(isobutyl)propan-1-amine hydrobromide

3-(Isobutylamino)propan-1-ol (46.0 g, 0.39 mol, 1 equiv.) was placed in a round bottomed flask and cooled to 0° C. (ice/water). To this mixture was carefully added HBr (294 mL in H₂O). The reaction mixture was heated to reflux for 16 h. The remaining HBr in H₂O was distilled off at 110° C. to provide the crude material as a brown solid, which was recrystallized from MeOH/Et₂O to afford the 3-bromo-N-(isobutyl)propan-1-amine hydrobromide as white crystals (47.9 g, 45%). ¹H NMR (500 MHz, D₂O) δ ppm 3.54 (t, J=6.5 Hz, 2H), 3.21 (t, J=8 Hz, 2H), 2.92 (d, J=7 Hz, 2H), 2.30-2.23 (m, 2H), 2.02 (sept, J=7 Hz, 1H), 0.99 (d, J=6.5 Hz, 6H). ¹³C NMR (125 MHz, D₂O) 55.0, 46.9, 30.0, 28.5, 25.8, 19.4. HRMS (ESI+) Calculated for C₇H₁₆BrN m/z 194.0544 (M+H), Obsd. 194.0546.

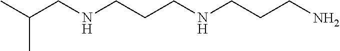

### Isobutyl Norspermidine [N¹-(3-Aminopropyl)-N³-(isobutyl)propane-1,3-diamine]

A round bottomed flask was charged with 1,3-diaminopropane (61.8 g, 0.83 mol, 10 equiv.), cooled to 0° C. (ice/water) and to this solution was added 3-bromo-N-isobutylpropan-1-amine hydrobromide salt (15.5 g, 0.08 mol, 1 equiv.) portionwise over the span of 1.5 h. The reaction mixture was left to warm and stirred for 12-16 h. Excess 1,3-diaminopropane was removed under reduced pressure, and the remaining semi-solid was taken up in 5% aqueous NaOH (100 mL) and extracted with 85:15 CHCl₃/i-PrOH (5×100 mL), dried over Na₂SO₄, filtered, and concentrated under reduced pressure. Purification was accomplished by fractional distillation (oil bath set at 210° C., distillate collected at 110° C.) to afford isobutyl norspermidine as a clear oil (7.4 g, 50%, best 71%). ¹H NMR (300 MHz, CDCl₃) δ ppm 2.76 (t, J=6.9 Hz, 2H), 2.70-2.63 (m, 6H), 2.39 (d, J=6.9 Hz, 2H), 1.80-1.59 (m, 5H), 1.49 (bs, 4H), 0.89 (d, J=6.6 Hz, 6H). ¹³C NMR (125 MHz, CDCl₃) δ ppm 58.4, 49.0, 48.9, 48.2, 40.8, 34.2, 30.7, 28.5, 20.9. HRMS (ESI+) Calculated for C₁₀H₂₅N₃ m/z 188.2127 (M+H), Obsd. 188.2123.

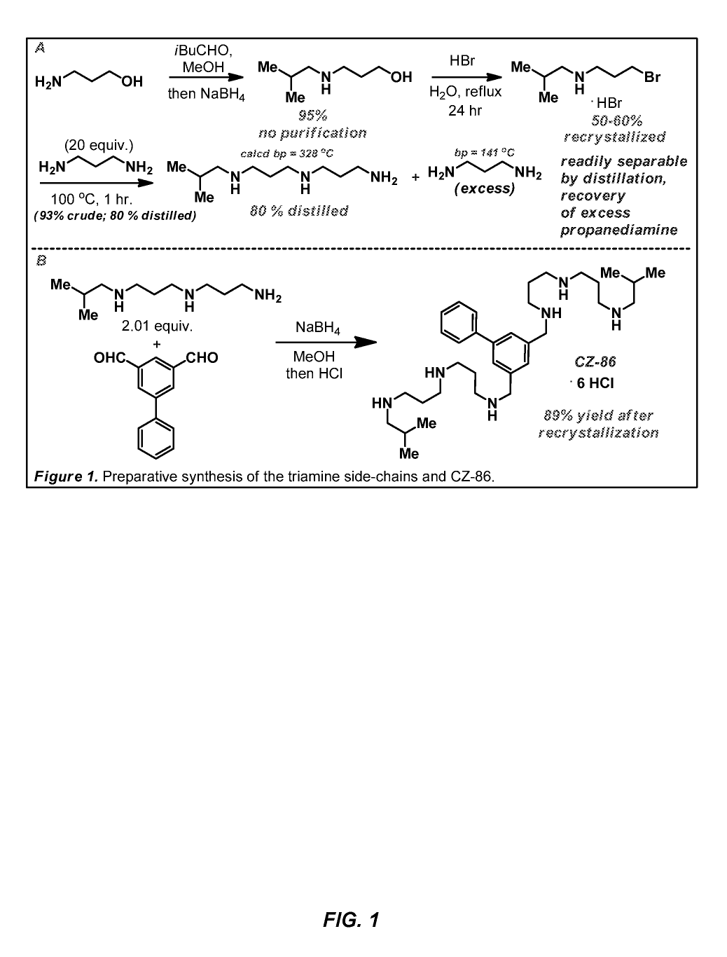

As exemplified in the synthesis of isobutyl norspermidine, this process is efficient and inexpensive, producing an N-alkyl polyamine in three steps for less than $0.30/g (FIG. 1A). The product can be further processed into oligomeric polyamines. A bis-reductive amination (cf. Baxter, E. W. & Reitz, A. B. Reductive Aminations of Carbonyl Compounds with Borohydride and Borane Reducing Agents. Org Reac 1, 59 (2004)) with a hydrophobic core and crystallization of the hexahydrochloride salt provides a di(N-alkyl polyamino) compound in four linear steps with no chromatography (judged to be >95% pure by 1H NMR and >97% pure by LCMS (UV)) as shown in FIG. 1B. This approach is cost-effective and amenable to commercial scale manufacturing required for environmental applications.

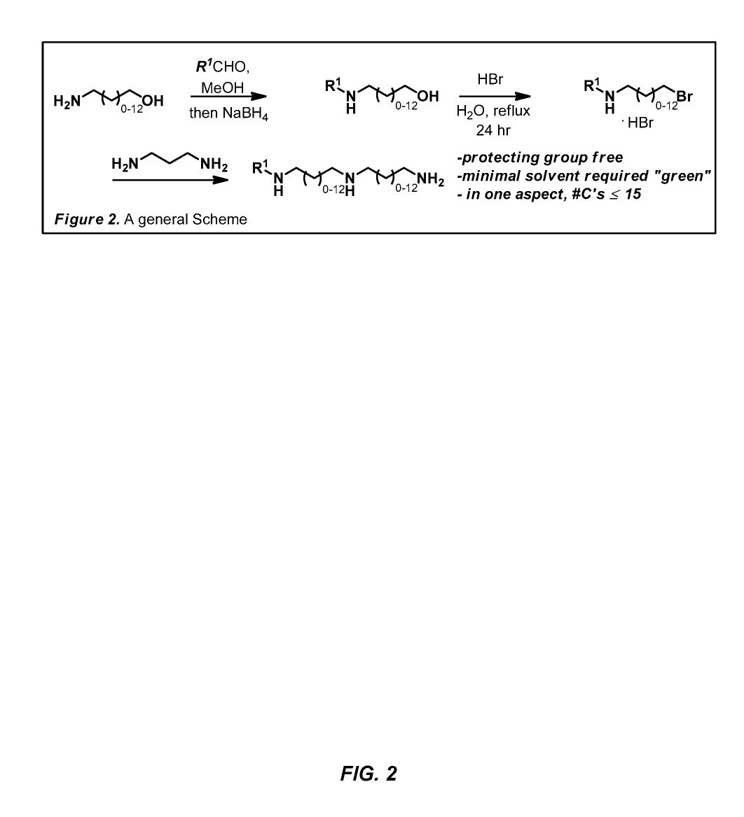

Thus, in a preferred aspect, the invention sets forth a process to produce N-alkyl polyamines as set forth in FIG. 2 in which the total number of carbons in the polyamine chain should be less than or equal to 15.

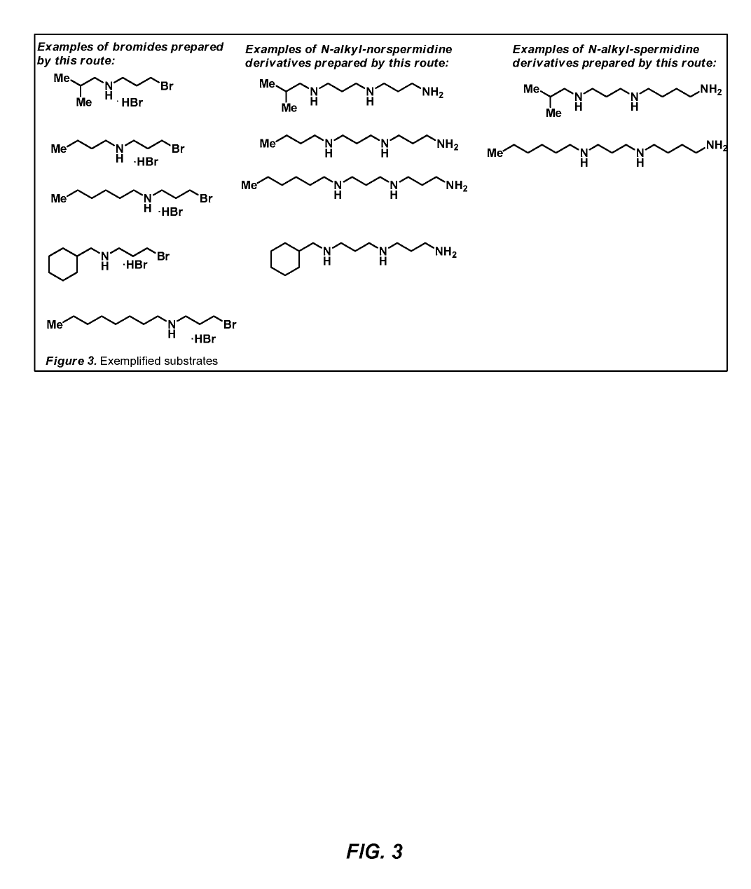

The exemplary norspermidine and spermidine derivatives as shown in FIG. 3 have been prepared according to the process discussed in this and the following examples.

### Example 2: Synthesis of N-(Bromoalkyl) Alkylamines

Selected N-(bromoalkyl)alkylamines were prepared according to the procedure of Example 1. In general, the substituted amino alcohol intermediates were used without further purification. If desired, vacuum distillation could be performed on the substituted amino alcohol intermediates to ensure purity.

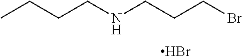

### N-(3-Bromopropyl)butan-1-amine hydrobromide

¹H NMR (500 MHz, D₂O) 6 ppm 3.55 (t, J=6.5 Hz, 2H), 3.22 (t, J=8.0 Hz, 2H), 3.07 (t, J=7.5 Hz, 2H), 2.29-2.23 (m, 2H), 1.70-1.64 (m, 2H), 1.39 (sext, J=7.5 Hz, 2H), 0.93 (t, J=7.5 Hz, 3H). ¹³C NMR (125 MHz, CDCl₃) δ ppm 48.2, 46.8, 29.8, 28.6, 27.8, 20.1, 13.6. HRMS (ESI+) Calculated for C₇H₁₆BrN m/z 194.0544 (M+H), Obsd. 194.0546. Yield (22% from 3-amino-1-propanol).

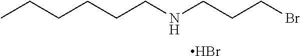

### N-(3-Bromopropyl)hexan-1-amine hydrobromide

¹H NMR (500 MHz, D₂O) 6 ppm 3.51 (t, J=6.0 Hz, 2H), 3.18 (t, J=7.0 Hz, 2H), 3.03 (t, J=7.5 Hz, 2H), 2.23 (quint, J=6.5 Hz, 2H), 1.65 (quint, J=8 Hz, 2H), 1.36-1.27 (m, 6H), 0.84 (t, J=7.0 Hz, 3H). ¹³C NMR (125 MHz, D₂O) δ ppm 48.0, 46.2, 30.6, 29.7, 28.5, 25.6, 25.5, 21.9, 13.4. HRMS (ESI+) Calculated for C₉H₂₀BrN m/z 222.0857 (M+H), Obsd. 222.0862. Yield (53% from 3-amino-1-propanol).

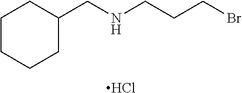 [SMILES: BrCCCNCC1CCCCC1 ; Cl]

### 3-Bromo-N-(cyclohexylmethyl)propan-1-amine hydrobromide

¹H NMR (500 MHz, D₂O) δ ppm 3.54 (t, J=6.0 Hz, 2H), 3.20 (t, J=7.5 Hz, 2H), 2.92 (d, J=7.0 Hz, 2H), 2.28-2.23 (m, 2H), 1.74-1.64 (m, 6H), 1.30-1.13 (m, 3H), 1.04-0.97 (m, 2H). ¹³C NMR (125 MHz, CDCl₃) δ ppm 54.2, 47.3, 34.5, 30.9, 29.9, 28.4, 25.8, 25.3. HRMS (ESI+) Calculated for C₁₀H₂₀BrN m/z 234.0857 (M+H), Obsd. 234.0862. Yield (41% from 3-amino-1-propanol).

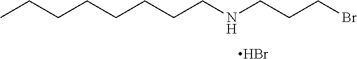

### N-(3-Bromopropyl)octan-1-amine hydrobromide

¹H NMR (500 MHz, D₂O) 6 ppm 3.53 (t, J=6.0 Hz, 2H), 3.20 (t, J=7.5 Hz, 2H), 3.05 (t, J=8.0 Hz, 2H), 2.27-2.22 (m, 2H), 1.67 (quint, J=7.0 Hz, 2H), 1.38-1.27 (m, 10H), 0.85 (t, J=7.0 Hz, 3H). ¹³C NMR (125 MHz, CDCl₃) δ ppm 48.3, 46.6, 31.7, 29.8, 29.1, 29.0, 28.6, 26.8, 25.8, 22.6, 14.1. HRMS (ESI+) Calculated for C₁₁H₂₄BrN m/z 250.1170 (M+H), Obsd. 250.1176. Yield (21% from 3-amino-1-propanol).

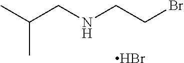

### N-(2-Bromoethyl)-2-methylpropan-1-amine, Hydrobromide Salt

¹H NMR (500 MHz, D₂O) δ ppm 3.73 (t, J=6.0 Hz, 2H), 3.55 (t, J=6.0 Hz, 2H), 2.98 (d, J=7.5 Hz, 2H), 2.06 (non, J=7.0 Hz, 1H), 1.01 (d, J=6.5 Hz, 6H). ¹³C NMR (125 MHz, D₂O) 54.6, 48.9, 25.9, 25.5, 19.3. HRMS (ESI+) Calculated for C₆H₁₄BrN m/z 180.0388 (M+H), Obsd. 180.0394. Yield (12%, two steps from ethanolamine).

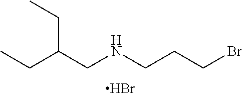

### N-(3-Bromopropyl)-2-ethylbutan-1-amine, Hydrobromide Salt

¹H NMR (500 MHz, D₂O) δ ppm 3.53 (t, J=6.0 Hz, 2H), 3.21 (t, J=7.5 Hz, 2H), 3.00 (d, J=7.0 Hz, 2H), 2.29-2.23 (m, 2H), 1.66 (sept, J=7.0 Hz, 1H), 1.39 (p, J=7.0 Hz, 4H), 0.87 (t, J=7.0 Hz, 6H). ¹³C NMR (125 MHz, D₂O) δ ppm 50.8, 46.8, 37.6, 29.6, 28.1, 22.4, 9.4. HRMS (ESI+) Calculated for C₉H₂₀BrN m/z 222.0857 (M+H), Obsd. 222.0856. Yield (45% from 3-amino-1-propanol).

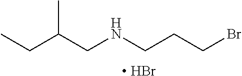

### N-(3-Bromopropyl)-2-methylbutan-1-amine, Hydrobromide Salt

¹H NMR (500 MHz, D₂O) δ ppm 3.51 (t, J=6.0 Hz, 2H), 3.18 (t, J=7.0 Hz, 2H), 3.00 (dd, J=6.5, 12 Hz, 1H), 2.86 (dd, J=8.0, 12.5 Hz, 1H), 2.26-2.21 (m, 2H), 1.78 (oct, J=7.0 Hz, 1H), 1.43-1.35 (m, 1H), 1.25-1.17 (m, 1H), 0.94 (d, J=6.5 Hz, 3H), 0.86 (t, J=7.5 Hz, 3H). ¹³C NMR (125 MHz, D₂O) δ ppm 53.3, 46.7, 31.7, 29.7, 28.2, 26.2, 16.1, 10.1. HRMS (ESI+) Calculated for C₈H₁₈BrN m/z 208.0701 (M+H), Obsd. 208.0703. Yield (36% from 3-amino-1-propanol).

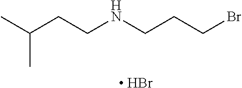

### N-(3-Bromopropyl)-3-methylbutan-1-amine, Hydrobromide Salt

¹H NMR (500 MHz, D₂O) δ ppm 3.54 (t, J=6.5 Hz, 2H), 3.20 (t, J=8.0 Hz, 2H), 3.08 (t, J=8.0 Hz, 2H), 2.27-2.22 (m, 2H), 1.66 (non, J=6.5 Hz, 1H), 1.58-1.54 (m, 2H), 0.91 (d, J=7.0 Hz, 6H). ¹³C NMR (125 MHz, D₂O) δ ppm 46.5, 46.3, 34.4, 29.9, 28.6, 25.4, 21.6. HRMS (ESI+) Calculated for C₈H₁₈BrN m/z 208.0701 (M+H), Obsd. 208.0705. Yield (42% from 3-Amino-1-propanol).

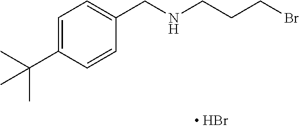 [SMILES: CC(C)(C)c1ccc(CNCCCBr)cc1]

### 3-Bromo-N-(4-(tert-butyl)benzyl)propan-1-amine, Hydrobromide Salt

¹H NMR (500 MHz, D₂O) δ ppm 7.54 (d, J=8.5 Hz, 2H), 7.41 (d, J=7.5 Hz, 2H), 4.20 (s, 2H), 3.66 (t, J=5.5 Hz, 2H), 3.12 (t, J=7.5 Hz, 2H), 1.91 (pent, J=6.5 Hz, 2H), 1.27 (s, 9H). ¹³C NMR (125 MHz, D₂O) δ ppm 153.3, 129.7, 127.7, 126.2, 58.9, 50.5, 44.6, 34.1, 30.4, 27.8. HRMS (ESI+) Calculated for C₁₄H₂₂BrN m/z 284.1014 (M+H), Obsd. 284.1017. Yield (33% from 3-amino-1-propanol).

### Example 3: Synthesis of N-Alkyl Norspermidines

Selected N-alkyl norspermidines were prepared according to the general procedure of Example 1 or a minor variant, which is disclosed below:

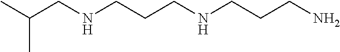

### N¹-(3-Aminopropyl)-N³-isobutylpropane-1,3-diamine

A round bottomed flask was charged with 1,3 diaminopropane (61.8 g, 0.83 mol, 10 equiv.), and cooled to 0° C. (ice/water). To this solution was added 3-bromo-N-isobutylpropan-1-amine hydrobromide (15.5 g, 0.08 mol, 1.0 equiv.) portionwise over the span of 1 h. The reaction mixture was left to warm and stirred for 12-16 h. Excess 1,3-diaminopropane was removed under reduced pressure, and the remaining semi-solid was taken up in 5% NaOH (100 mL) and extracted with CHCl₃ (2×100 mL), dried over Na₂SO₄, filtered, and concentrated under reduced pressure. Purification was accomplished by fractional distillation (oil bath set at 210° C., distillate collected at 110° C.) to afford pure N¹-(3-aminopropyl)-N³-isobutylpropane-1,3-diamine as a clear oil (7.4 g, 50%). ¹H NMR (300 MHz, CDCl₃) δ ppm 2.76 (t, J=6.9 Hz, 2H), 2.70-2.63 (m, 6H), 2.39 (d, J=6.9 Hz, 2H), 1.80-1.59 (m, 5H), 1.49 (bs, 4H), 0.89 (d, J=6.6 Hz, 6H). ¹³C NMR (125 MHz, CDCl₃) δ ppm 58.4, 49.0, 48.9, 48.2, 40.8, 34.2, 30.7, 28.5, 20.9.

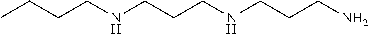

### N¹-(3-Aminopropyl)-N³-butylpropane-1,3-diamine

¹H NMR (500 MHz, CDCl₃) δ ppm 2.64-2.46 (m, 10H), 1.56-1.49 (m, 4H), 1.37-1.30 (m, 2H), 1.27-1.18 (m, 6H), 0.81-0.77 (m, 3H). ¹³C NMR (125 MHz, CDCl₃) δ ppm 50.1, 48.9, 48.8, 48.1, 40.8, 34.2, 32.5, 30.7, 20.7, 14. HRMS (ESI+) Calculated for C₁₀H₂₅N₃ m/z 188.2127 (M+H), Obsd. 188.2126. Yield (45%, 7.01 g).

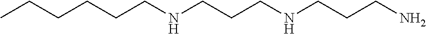

### N¹-(3-Aminopropyl)-N³-hexylpropane-1,3-diamine (Hexyl Norspermidine)

¹H NMR (500 MHz, CDCl₃) δ ppm 2.75 (t, J=7 Hz, 2H), 2.65 (td, J=2.5 Hz, 7.0 Hz, 6H), 2.57 (t, J=7.5 Hz, 2H), 1.69-1.59 (m, 4H), 1.47-1.43 (m, 2H), 1.32-1.22 (m, 6H), 1.13 (bs, 4H), 0.87 (t, J=6.5 Hz, 3H). ¹³C NMR (125 MHz, CDCl₃) δ ppm 50.3, 48.9, 48.8, 48.1, 40.8, 33.7, 32.0, 30.2, 27.3, 22.8, 14.3. HRMS (ESI+) Calculated for C₁₂H₂₉N₃ m/z 216.2440 (M+H), Obsd. 216.2443. Yield (55%, 12.74 g).

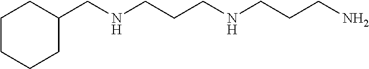 [SMILES: NCCCNCCCNCC1CCCCC1]

### N¹-(3-Aminopropyl)-N₃-(cyclohexylmethyl)propane-1,3-diamine (Cyclohexylmethyl Norspermidine)

¹H NMR (500 MHz, CDCl₃) δ ppm 2.75 (t, J=7.0 Hz, 2H), 2.66-2.61 (m, 6H), 2.40 (d, J=7.0 Hz, 2H), 1.72-1.59 (m, 9H), 1.47-1.38 (m, 1H), 1.27-1.03 (m, 7H), 0.91-0.84 (m, 2H). ¹³C NMR (125 MHz, CDCl₃) δ ppm 57.2, 49.0, 48.2, 40.8, 38.2, 34.2, 31.7, 30.7, 26.9, 26.3. HRMS (ESI+) Calculated for C₁₃H₂₉N₃ m/z 228.2440 (M+H), Obsd. 228.2439. Yield (38%, 3.43 g).

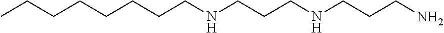 [SMILES: CCCCCCCCNCCCNCCCN]

### N-(3-Aminopropyl)-N³-octylpropane-1,3-diamine (octyl norspermidine)

¹H NMR (500 MHz, CDCl₃) δ ppm 2.75 (t, J=7.0 Hz, 2H), 2.66 (t, J=7.0 Hz, 6H), 2.57 (t, J=7.5 Hz, 2H), 1.69-1.60 (m, 4H), 1.49-1.42 (m, 2H), 1.27 (s, 10H), 1.04 (bs, 4H), 0.87 (t, J=6.5 Hz, 3H). ¹³C NMR (125 MHz, CDCl₃) 50.1, 48.7, 48.6, 47.9, 40.5, 33.6, 31.8, 30.1, 30.0, 29.5, 29.2, 27.4, 22.6, 14.1. HRMS (ESI+) Calculated for C₁₄H₃₃N₃ m/z 244.2753 (M+H), Obsd. 244.2756. Yield (43%, 6.70 g).

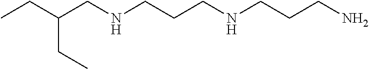

### N-(3-Aminopropyl)-N³-(2-ethylbutyl)propane-1,3-diamine (Gem-Diethyl Norspermidine)

¹H NMR (300 MHz, CDCl₃) δ ppm 2.64 (t, J=6.9 Hz, 2H), 2.58-2.51 (m, 6H), 2.36 (d, J=5.4 Hz, 2H), 1.53 (sept, J=6.6 Hz, 4H), 1.25-1.15 (m, 5H), 0.97 (bs, 4H), 0.74 (t, J=6.9 Hz, 6H). ¹³C NMR (75 MHz, CDCl₃) δ ppm 53.1, 49.1, 49.0, 48.1, 41.0, 40.7, 34.2, 30.5, 24.2, 11.1. HRMS (ESI+) Calculated for C₁₂H₂₉N₃ m/z 216.2440 (M+H), Obsd. 216.2439. Yield (56%, 11.92 g).

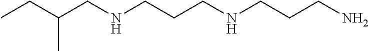

### N¹-(3-Aminopropyl)-N³-(2-methylbutyl)propane-1,3-diamine

¹H NMR (500 MHz, CDCl₃) δ ppm 2.71 (t, J=7.0 Hz, 2H), 2.63-2.58 (m, 6H), 2.46 (dd, J=6.0, 11.5 Hz, 1H), 2.31 (dd, J=7.5, 12.0 Hz, 1H), 1.65-1.55 (m, 4H), 1.51-1.42 (m, 1H), 1.39-1.30 (m, 1H), 1.12-1.05 (m, 5H), 0.85-0.82 (m, 6H). ¹³C NMR (125 MHz, CDCl₃) δ ppm 56.5, 49.0, 48.1, 40.7, 34.9, 34.2, 30.6, 27.7, 17.8, 11.5. HRMS (ESI+) Calculated for C₁₁H₂₇N₃ m/z 202.2283 (M+H), Obsd. 202.2287. Yield (51%, 10.06 g).

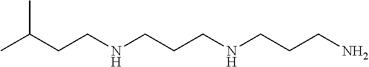

### N¹-(3-Aminopropyl)-N³-isopentylpropane-1,3-diamine (Isoamyl Norspermidine)

¹H NMR (500 MHz, CDCl₃) δ ppm 2.75 (t, J=6.5 Hz, 2H), 2.66 (t, J=12.0 Hz, 6H), 2.58 (t, J=13.0 Hz, 2H), 1.72-1.55 (m, 5H), 1.39-1.32 (m, 6H), 0.88 (d, J=11.0 Hz, 6H). ¹³C NMR (125 MHz, CDCl₃) δ ppm 48.7, 48.2, 47.9, 40.5, 39.2, 33.9, 30.4, 26.1, 22.6. HRMS (ESI+) Calculated for C₁₁H₂₇N₃ m/z 202.2283 (M+H), Obsd. 202.2283. Yield (61%, 16.89 g).

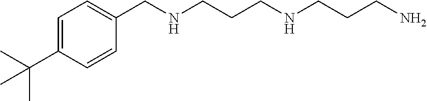 [SMILES: CC(C)(C)c1ccc(CNCCCNCCCN)cc1]

### N-(3-Aminopropyl)-N³-(4-(tert-butyl)benzyl)propane-1,3-diamine (Tert-Butyl Benzyl Norspermidine)

¹H NMR (500 MHz, CDCl₃) δ ppm 7.34 (d, J=8.0 Hz, 2H), 7.23 (d, J=8.0 Hz, 2H), 3.74 (s, 2H), 2.75 (t, J=7.0 Hz, 2H), 2.71-2.64 (m, 6H), 1.70 (pent, J=7.0 Hz, 2H), 1.62 (pent, J=7.0 Hz, 2H), 1.43 (bs, 4H), 1.31 (s, 9H). ¹³C NMR (125 MHz, CDCl₃) δ ppm 149.8, 137.2, 127.9, 125.3, 53.6, 48.4, 47.8, 47.7, 40.2, 34.5, 32.8, 31.4, 29.9. LRMS Calculated for C₁₇H₃₁N₃ m/z 278.2596 [M+H]⁺, Obsd. 278.2594. Yield (38%, 5.16 g).

### Example 4: Synthesis of N-Alkyl Spermidines

Selected N-alkyl spermidines were prepared according to the general procedures of Example 1 or 3:

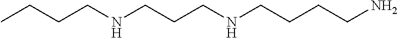

### N¹-(3-Butylamino)propyl)butane-1,4-diamine (Butyl Spermidine)

¹H NMR (300 MHz, CDCl₃) δ: 2.67-2.51 (m, 10H), 1.62 (pent, J=7.2 Hz, 2H), 1.52-1.34 (m, 6H), 1.30 (pent, J=7.2 Hz, 2H), 0.98 (bs, 4H), 0.86 (t, J=7.2 Hz, 3H). ¹³C NMR (75 MHz, CDCl₃) δ ppm 50.2, 50.1, 48.8, 48.8, 42.4, 32.5, 31.9, 30.8, 27.7, 20.7, 14.2. HRMS (ESI+) Calculated for C₁₁H₂₇N₃ m/z 202.2283 (M+H), Obsd. 201.2284. Yield (48%, 5.28 g).

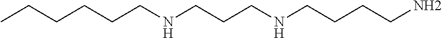 [SMILES: CCCCCCNCCCNCCCCN]

### N¹-(3-(Hexylamino)propyl)butane-1,4-diamine (Hexyl Spermidine)

¹H NMR (500 MHz, CDCl₃) δ: 2.80 (bs, 4H), 2.65-2.50 (m, 10H), 1.65-1.59 (m, 2H), 1.46-1.38 (m, 6H), 1.25-1.17 (m, 6H), 0.81 (t, J=7.0 Hz, 3H). ¹³C NMR (125 MHz, CDCl₃) δ ppm 50.2, 49.9, 48.6, 42.0, 31.9, 31.3, 30.2, 30.1, 27.4, 27.2, 22.7, 14.2. HRMS (ESI+) Calculated for C₁₃H₃₁N₃ m/z 230.2596 (M+H), Obsd. 230.2601. Yield (51%, 4.54 g).

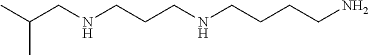

### N¹-(3-(Isobutylamino)propyl)butane-1,4-diamine (Isobutyl Spermidine)

¹H NMR (500 MHz, CDCl₃) δ ppm 2.66-2.54 (m, 8H), 2.35-2.33 (m, 2H), 1.70-1.59 (m, 3H), 1.49-1.40 (m, 4H), 0.98 (bs, 4H), 0.85-0.83 (n, 6H). ¹³C NMR (125 MHz, CDCl₃) δ ppm 58.4, 50.1, 48.9, 48.9, 42.4, 31.9, 30.6, 28.4, 27.7, 20.8. HRMS (ESI+) Calculated for C₁₁H₂₇N₃ m/z 202.2283 (M+H), Obsd. 202.2284. Yield (53%, 7.95 g).

### Example 5: Synthesis of N-Alkyl Polyamines

Some other exemplary N-alkyl polyamines were prepared according to the general procedures of Examples 1 or 3:

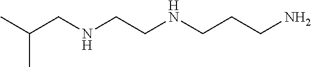

### N¹-(2-(Isobutylamino)ethyl)propane-1,3-diamine

¹H NMR (500 MHz, CDCl₃) δ ppm 2.71 (t, J=7.0 Hz, 2H), 2.64 (s, 4H), 2.62 (t, J=7.0 Hz, 2H), 2.33 (d, J=7.0 Hz, 2H), 1.97 (bs, 4H), 1.66 (sept, J=6.5 Hz, 1H), 1.58 (pent, J=7.0 Hz, 2H), 0.82 (d, J=6.5 Hz, 6H). ¹³C NMR (125 MHz, CDCl₃) δ ppm 58.1, 49.5, 49.4, 47.9, 40.6, 33.4, 28.4, 20.8. HRMS (ESI+) Calculated for C₉H₂₃N₃ m/z 174.1970 (M+H), Obsd. 174.1977. Yield (42%, 4.04 g).

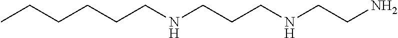

### N¹-(2-Aminoethyl)-N³-hexylpropane-1,3-diamine

¹H NMR (500 MHz, CDCl₃) δ 2.68 (t, J=6.0 Hz, 2H), 2.58-2.54 (m, 6H), 2.47 (t, J=7.0 Hz, 2H), 1.57 (pent, J=7.0 Hz, 2H), 1.39-1.31 (m, 2H), 1.22-1.07 (m, 10H), 0.77 (t, J=7.5 Hz, 3H). ¹³C NMR (125 MHz, CDCl₃) δ ppm 52.7, 50.2, 48.6, 48.4, 41.8, 31.8, 30.5, 30.2, 27.1, 22.6, 14.1. HRMS (ESI+) Calculated for C₁₁H₂₇N₃ m/z 202.2283 (M+H), Obsd. 202.2291. Yield (47%, 3.23 g).

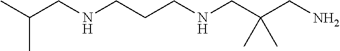

### N¹-(3-(Isobutylamino)propyl)-2,2-dimethylpropane-1,3-diamine

¹H NMR (500 MHz, CDCl₃) δ ppm 2.63 (t, J=7.0 Hz, 4H), 2.50 (s, 2H), 2.39-2.37 (m, 4H), 1.77-1.62 (m, 3H), 1.02 (bs, 4H), 0.88 (d, J=7.0 Hz, 6H), 0.84 (s, 6H). ¹³C NMR (125 MHz, CDCl₃) δ ppm 59.1, 58.5, 51.7, 49.9, 49.0, 35.6, 30.5, 28.5, 23.9, 20.9. HRMS (ESI+) Calculated for C₁₂H₂₉N₃ m/z 216.2440 (M+H), Obsd. 216.2444. Yield (55%, 8.60 g).

### Example 6: Synthesis of N,N-Dialkyl Polyamines

Selected N,N-dialkyl polyamines were prepared according to the general procedure set forth below:

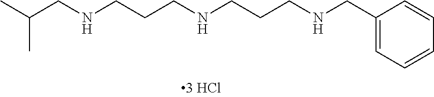 [SMILES: CC(C)CNCCCNCCCNCc1ccccc1]

### N¹-Benzyl-N³-(3-(isobutylamino)propyl)propane-1,3-diamine, Hydrochloride Salt

Benzaldehyde (0.16 g, 1.56 mmol, 1 equiv.) was added dropwise to a cooled solution (0° C.) of isobutyl norspermidine (0.29 g, 1.56 mmol, 1 equiv.) in methanol (5 mL), and the reaction was left to stir for 16 h. Sodium borohydride (0.24 g, 6.24 mmol, 4 equiv.) was then added portionwise, and the reaction mixture was stirred for 1 h. The excess methanol was evaporated and the crude solid was partitioned between ethyl acetate (50 mL) and 10% aq. NaOH (1×50 mL). The aqueous layer was then back extracted with ethyl acetate (1×50 mL) dried over Na₂SO₄, and evaporated to afford the crude free base, which was carried forward without further purification. The crude free base was acidified with HCl in MeOH (50 mL) and then placed at 0° C. for 1 h. The resulting precipitate was filtered and dried to afford the pure HCl salt as a white solid (52%). ¹H NMR (500 MHz, D₂O) δ ppm 7.51 (s, 5H), 4.28 (s, 2H), 3.23-3.14 (m, 8H), 2.93 (d, J=6.5 Hz, 2H), 2.19-2.12 (m, 4H), 2.02 (sept, J=6.5 Hz, 1H), 1.00 (d, J=7.0 Hz, 6H). ¹³C NMR (125 MHz, D₂O) δ ppm 130.6, 130.1, 130.0, 129.5, 55.1, 51.4, 45.0, 44.9, 44.8, 44.0, 25.8, 22.8, 22.7, 19.3. Yield (52%, 0.31 g).

The following compounds were prepared similarly to N¹-benzyl-N³-(3-(isobutylamino)propyl)propane-1,3-diamine, hydrochloride salt:

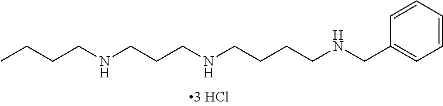 [SMILES: CCCCNCCCNCCCCNCc1ccccc1]

### N¹-Benzyl-N³-(3-(butylamino)propyl)propane-1,3-diamine, Hydrochloride Salt

¹H NMR (500 MHz, D₂O) δ ppm 7.52-7.49 (m, 5H), 4.28 (s, 2H), 3.22-3.13 (m, 8H), 3.07 (t, J=7.5 Hz, 4H), 2.18-2.09 (m, 4H), 1.66 (pent, J=7.5 Hz, 2H), 1.39 (hex, J=7.5 Hz, 2H), 0.93 (t, J=7.0 Hz, 3H). ¹³C NMR (125 MHz, D₂O) δ ppm 130.5, 130.0, 130.0, 129.5, 51.4, 47.8, 44.8, 44.8, 44.4, 44.0, 27.7, 22.8, 19.3, 12.9. HRMS (ESI+) Calculated for C₁₈H₃₃N₃ m/z 292.2753 (M+H), Obsd. 292.2753. Yield (45%, 0.82 g).

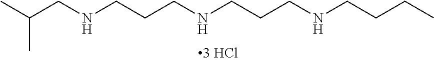 [SMILES: CCCCNCCCNCCCNCC(C)C]

### N¹-Butyl-N³-(3-(Isobutylamino)propyl)propane-1,3-diamine, Hydrochloride Salt

¹H NMR (500 MHz, D₂O) δ ppm 3.19-3.13 (m, 8H), 3.06 (t, J=7.5 Hz, 2H), 2.92 (d, J=7.5 Hz, 2H), 2.16-2.08 (m, 4H), 2.01 (sept, J=7.0 Hz, 1H), 1.65 (pent, J=7.5 Hz, 2H), 1.38 (hex, J=7.0 Hz, 2H), 0.98 (d, J=6.5 Hz, 6H), 0.91 (t, J=8.0 Hz, 3H). ¹³C NMR (125 MHz, D₂O) δ ppm 55.1, 47.8, 44.9, 44.8, 44.4, 27.7, 25.8, 22.8, 22.7, 19.3, 19.2, 12.9. HRMS (ESI+) Calculated for C₁₄H₃₃N₃ m/z 244.2753 (M+H), Obsd. 244.2750. Yield (45%, 0.24 g).

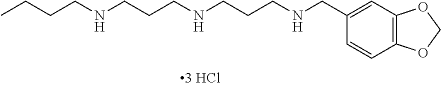 [SMILES: CCCCNCCCNCCCNCc1ccc2c(c1)OCO2]

### N¹-(Benzo[d][1,3]dioxol-5-ylmethyl)-N³-(3-(butylamino)propyl)propane-1,3-diamine, Hydrochloride Salt

¹H NMR (500 MHz, D₂O) δ ppm 6.98-6.92 (m, 3H), 6.00 (s, 2H), 4.16 (s, 2H), 3.17-3.11 (m, 8H), 3.05 (t, J=7.0 Hz, 2H), 2.15-2.08 (m, 4H), 1.64 (pent, J=7.5 Hz, 2H), 1.37 (hex, J=7.0 Hz, 2H), 0.90 (t, J=7.5 Hz, 3H). ¹³C NMR (125 MHz, D₂O) δ ppm 148.4, 147.9, 124.4, 124.2, 110.1, 109.1, 108.1, 51.2, 47.8, 44.8, 44.8, 43.8, 27.7m, 22.9, 19.3, 12.9. HRMS (ESI+) Calculated for C₁₈H₃₁N₃O₂ m/z 322.2511 (M+H), Obsd. 322.2494. Yield (55%, 0.17 g).

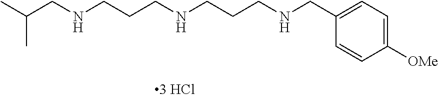 [SMILES: COc1ccc(CNCCCNCCCNCC(C)C)cc1]

### N¹-Isobutyl-N³-(3-((4-methoxybenzyl)amino)propyl)propane-1,3-diamine, Hydrochloride Salt

¹H NMR (500 MHz, D₂O) δ ppm 7.44 (d, J=8.0 Hz, 2H), 7.05 (d, J=9.0 Hz, 2H), 4.22 (s, 2H), 3.84 (s, 3H), 3.19-3.14 (m, 8H), 2.93 (d, J=7.0 Hz, 2H), 2.18-2.11 (m, 4H), 2.02 (sept, J=7.0 Hz, 1H), 1.00 (d, J=7.0 Hz, 6H). ¹³C NMR (125 MHz, D₂O) δ ppm 160.0, 131.8, 123.0, 114.8, 55.6, 55.1, 50.9, 45.0, 44.8, 43.8, 25.8, 22.8, 22.7, 19.3. HRMS (ESI+) Calculated for C₁₈H₃₃N₃O m/z 308.2702 (M+H), Obsd. 308.2702. Yield (60%, 0.58 g).

1. A method of preparing an N-alkyl polyamine, wherein the method comprises the steps:
reacting an aminoalkyl alkylating agent in a reaction mixture comprising an excess amount of a polyaminoalkane to produce a N-alkyl polyamine, wherein the aminoalkyl alkylating agent comprises (i) a secondary or tertiary amino group and (ii) a halo or aldehyde group; and wherein the N-alkyl polyamine has from 5 to 30 carbon atoms; and
distilling a crude product comprising the N-alkyl polyamine to provide a purified N-alkyl polyamine.

2. (canceled)

3. (canceled)

4. The method of claim 1, wherein the N-alkyl polyamine has from 5 to 20 carbon atoms.

5. (canceled)

6. The method of claim 4, wherein the N-alkyl polyamine has from 5 to 15 carbon atoms.

7. The method of claim 6, wherein the N-alkyl polyamine has from 10 to 15 carbon atoms.

8. (canceled)

9. (canceled)

10. (canceled)

11. (canceled)

12. The method of claim 1, wherein the step of reacting the aminoalkyl alkylating agent is performed at a temperature from −10° C. to 100° C.

13. (canceled)

14. (canceled)

15. The method of claim 1, wherein the step of reacting the aminoalkyl alkylating agent is performed at a temperature from 0° C. to 60° C.

16. (canceled)

17. The method of claim 15, wherein the step of reacting the aminoalkyl alkylating agent is performed at a temperature from 10° C. to 25° C.

18. The method of claim 1, wherein the step of distilling the crude product is performed at below atmospheric pressure.

19. The method of claim 18, wherein the step of distilling the crude product is performed at a pressure from 10 mm Hg to 25 mm Hg.

20. The method of claim 18, wherein the step of distilling the crude product is performed at a pressure from 1 mm Hg to 10 mm Hg.

21. (canceled)

22. The method of claim 1, wherein the step of reacting the aminoalkyl alkylating agent includes no added solvent.

23. (canceled)

24. The method of claim 1, wherein the aminoalkyl alkylating agent is of the formula 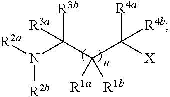
wherein each R substituent is an independently selected hydrogen, alkyl, alkoxy alkenyl, or alkynyl group; wherein at least one R² substituent is not hydrogen; and wherein X is —CHO or a halo group.

25. (canceled)

26. (canceled)

27. (canceled)

28. (canceled)

29. (canceled)

30. (canceled)

31. (canceled)

32. (canceled)

33. (canceled)

34. The method of claim 24, wherein X is a halo group.

35. The method of claim 1, wherein the aminoalkyl alkylating agent is selected from the group consisting of an N-alkyl propylene halide or aldehyde, an N-alkyl butylene halide or aldehyde, an N-alkyl pentylene halide or aldehyde, an N-alkyl ethylene halide or aldehyde, and an N-alkyl hexylene halide or aldehyde.

36. The method of claim 1, wherein the N-alkyl group is butyl, isobutyl, hexyl, (cyclohexyl)methyl, octyl, isopropyl, methyl, ethyl, cyclohexyl, prenyl, propargyl, or cyclopropyl.

37. (canceled)

38. The method of claim 1, wherein the aminoalkyl alkylating agent is a crystalline salt with a halide counterion.

39. The method of claim 1, wherein the polyaminoalkane is spermidine.

40. (canceled)

41. The method of claim 40 of claim 1, wherein the excess amount is at least 5 equivalents.

42. The method of claim 41, wherein the excess amount is at least 8 equivalents.

43. The method of claim 42, wherein the excess amount is at least 12 equivalents.

44. (canceled)

45. The method of claim 44, wherein the excess amount is at least 20 equivalents.

46. (canceled)

47. (canceled)

48. (canceled)

49. (canceled)

50. (canceled)

51. (canceled)

52. (canceled)

53. The method of claim 1, wherein the method further comprises a step of distilling the crude product to produce a purified diaminoalkane.

54. The method of claim 53, wherein the method further comprises reusing the purified diaminoalkane as a substrate for alkylation.

55. The method of claim 1, wherein the method further comprises a step of reacting an aminoalkyl alcohol precursor to produce the aminoalkyl alkylating agent as a crystalline salt.

56. The method of claim 55, wherein the method further comprises a step of reacting a primary aminoalkyl alcohol with an alkyl aldehyde or a cycloalkylmethyl aldehyde to produce the aminoalkyl alcohol precursor.

57. The method of claim 55, wherein the method further comprises a step of reacting a secondary aminoalkyl alcohol with an alkyl aldehyde or a cycloalkylmethyl aldehyde to produce the aminoalkyl alcohol precursor.

58. The method of claim 53, wherein the method further comprises a step of reacting the purified N-alkyl polyamine with an aldehyde or halide to produce an oligomeric polyamine.

59. The method of claim 53, wherein the method further comprises a step of reacting the purified N-alkyl polyamine with a polyaldehyde or polyhalide to produce an oligomeric polyamine.

60. The method of claim 59, wherein the oligomeric polyamine is a polyamine compound selected from the group consisting of 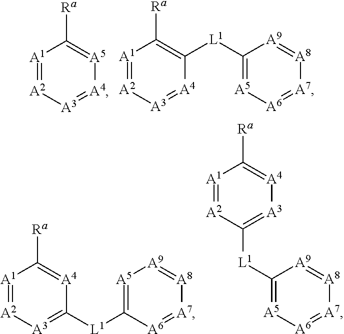
and a salt thereof;
wherein:
each R^{a} is a member independently selected from the group consisting of 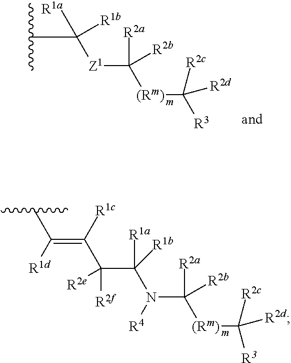
A¹, A², A³, A⁴, A⁵, A⁶, A⁷, A⁸, and A⁹ are each an Aⁿ member independently selected from the group consisting of N, CR^{a}, and CR⁵; or, alternatively, a pair of adjacent Aⁿ members join to form an independently selected aryl, cycloalkyl, heterocyclyl, or heterocycloaryl ring that is fused with an Aⁿ ring at the pair's Aⁿ ring positions; wherein at least one Aⁿ member and at most five Aⁿ members are an independently selected CR^{a};
each R^{1a}, R^{1b}, R^{1c}, and R^{1d} is a member independently selected from the group consisting of hydrogen, fluoro, alkyl, and fluoroalkyl; or, alternatively, an R^{1a} and an R^{1b} join to form an oxo group;
each R^{2a}, R^{2b}, R^{2c}, R^{2d}, R^{2e}, and R^{2f} is a member independently selected from the group consisting of hydrogen, alkyl, fluoroalkyl, alkenyl, alkynyl, aryl, heteroaryl, arylalkyl, and heteroarylalkyl; alternatively, a pair of R² members from the same R^{a} group independently selected from R^{2a} and R^{2b}, R^{2c} and R^{2d}, or R^{2e} and R^{2f} join to form a member independently selected from the group consisting of spirocycloalkyl, spiroheterocycyl, and oxo; or, alternatively, an R^{2a} and an R^{2c} from the same R^{a} group join to form a ring independently selected from the group consisting of cycloalkyl and heterocycyl;
each R^{m} is a member independently selected from the group consisting of —CR^{2a}R^{2b}—, —CR^{2c}R^{2d}—, —C(R^{2a})═(R^{2b})—, —CC—, and —C(R^{2a})(R^{2b})-L²-C(R^{2c})(R^{2d})—;
each m is an integer independently selected from 1 to 20;
each L¹ and L² is a member independently selected from the group consisting of a bond, —O—, —C(O)O—, —NR⁴—, —NR⁴C(O)—, and —C(O)NR⁴—;
each R³ is a member independently selected from the group consisting of —Z¹—R⁴, —Z¹—Y¹—R⁴, —Z¹—Y¹—Y²—R⁴, and —Z¹—Y¹—Y²—Y³—R⁴;
each R⁴ is a member independently selected from the group consisting of hydrogen, alkyl, fluoroalkyl, alkenyl, alkynyl, aryl, cycloalkyl, heteroaryl, arylalkyl, cycloalkylalkyl, and heteroarylalkyl; or, alternatively, for an —N(R⁴)₂ group, one of the two R⁴ in the group is a member selected from the group consisting of —(CO)OR^{6a}—, —(CO)N(R^{6a})(R^{6b}), and —C(NR^{6a})N(R^{6b})(R^{6c}); or, alternatively, for an —N(R⁴)₂ group, the two R⁴ groups join to form a heterocyclic ring;
each R⁵ is a member independently selected from the group consisting of hydrogen, alkyl, hydroxyl, alkoxy, aminoalkoxy, alkylamino, alkylaminoalkoxy, alkenyl, alkynyl, aryl, aryloxy, arylamino, cycloalkyl, cycloalkoxy, cycloalkylalkoxy, cycloalkylamino, cycloalkylalkylamino, heterocyclyl, heterocycyloxy, heterocycylamino, halo, haloalkyl, fluoroalkyloxy, heteroaryl, heteroaryloxy, heteroarylamino, arylalkyl, arylalkyloxy, arylalkylamino, heteroarylalkyl, heteroarylalkyloxy, heteroarylalkylamino, hydroxyalkyl, aminoalkyl, and alkylaminoalkyl;
each Y¹, Y², and Y³ is an independently selected group of Formula IA: 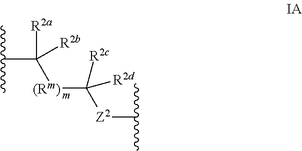
each Z¹ and Z² is a member independently selected from the group consisting of —N(R⁴)— and —O—; and
each R^{6a}, R^{6b}, and R^{6c} is a member independently selected from the group consisting of hydrogen, alkyl, fluoroalkyl, alkenyl, alkynyl, aryl, heteroaryl, cycloalkyl, arylalkyl, heteroarylalkyl, and cycloalkylalkyl; or, alternatively, two R⁶ⁿ members R^{6a} and R^{6b} or R^{6a} and R^{6c} join to form a heterocycyl ring; and
wherein the polyamine compound comprises at least two primary or secondary amino groups.

61. The method of claim 60, wherein the oligomeric polyamine is 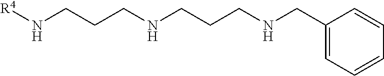
or a salt thereof; and
wherein R⁴ is hydrogen or alkyl.

62. The method of claim 60, wherein the oligomeric polyamine is 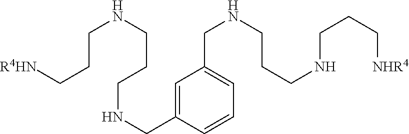
or a salt thereof; and
wherein R⁴ is hydrogen or alkyl.

63. The method of claim 60, wherein the oligomeric polyamine is 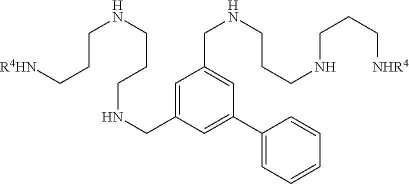
or a salt thereof; and
wherein R⁴ is hydrogen or alkyl.
